(19) United States
(12) Patent Application Publication
(10) Pub. No.: US 2024/0201157 A1
(43) Pub. Date: Jun. 20, 2024

(54) GAS ANALYSIS DEVICE, FLUID CONTROL SYSTEM, PROGRAM FOR GAS ANALYSIS, AND GAS ANALYSIS METHOD

(71) Applicant: HORIBA STEC, Co., Ltd., Kyoto (JP)

(72) Inventor: Motonobu TAKAHASHI, Kyoto (JP)

(73) Assignee: HORIBA STEC, Co., Ltd., Kyoto (JP)

(21) Appl. No.: 18/288,097

(22) PCT Filed: Feb. 14, 2022
(86) PCT No.: PCT/JP2022/005599

§ 371 Date: Oct. 24, 2023

(30) Foreign Application Priority Data
Apr. 30, 2021 (JP) 2021-078046

(51) Int. Cl. G01N 33/00; G01N 1/02; G01N 33/18

(52) U.S. Cl. CPC G01N 33/0067; G01N 1/02; G01N 33/18; G01N 2001/028; G01N 2033/0068

(57) ABSTRACT

A gas analysis device for analyzing a compound gas and H₂O gas produced in a main reaction in which an aqueous solution including a compound and water is vaporized, includes a first concentration calculating unit that calculates a concentration of the compound gas, a second concentration calculating unit that calculates a concentration of the H₂O gas, an analysis unit that compares a first actual concentration which is the concentration of the compound gas calculated by the first concentration calculating unit with a first ideal concentration which is the concentration of the compound gas in case that the main reaction proceeds ideally, and that compares a second actual concentration which is the concentration of the H₂O gas calculated by the second concentration calculating unit with a second ideal concentration which is the concentration of the H₂O gas in case that the main reaction proceeds ideally and an output unit.

### TECHNICAL FIELD

This invention relates to a gas analysis device, a fluid control system, a program for gas analysis, and a gas analysis method.

### BACKGROUND ART

For example, a hydrogen peroxide gas consisting of vaporized hydrogen peroxide is sometimes used in a cleaning process of semiconductor manufacturing processes or in a sterilization process of medical instruments. Concretely, the hydrogen peroxide gas is generated by vaporizing an aqueous solution of liquid hydrogen peroxide mixed with water.

As shown in Patent document 1, there is a system that uses the hydrogen peroxide gas, which is provided with a concentration monitor that detects the concentration of the hydrogen peroxide gas. In accordance with this arrangement, it is possible to monitor whether the concentration of the supplied hydrogen peroxide gas is the desired concentration or not, in other words, whether or not the concentration of the supplied hydrogen peroxide gas is the ideal concentration that is obtained in case that the vaporization of the above-mentioned aqueous solution proceeded ideally.

However, even if the concentration monitor is arranged as mentioned above, in case that there is a difference between the actual concentration detected by the concentration monitor and the desired ideal concentration, it is not possible to identify the cause of the difference. This is because, for example, various factors can be conceived that may cause the actual concentration to be lower than the ideal concentration, such as the above-mentioned aqueous solution vaporization does not proceed ideally, or a complex of side reactions such as liquefaction or decomposition of the hydrogen peroxide gas.

As a result of this, even if it is found that there is a difference between the actual concentration measured by the concentration monitor and the ideal concentration, it is not possible to determine how to deal with the difference, and the difference will have to be filled by trial and error.

This problem is not limited to the hydrogen peroxide gas, but also is occurring when compounds such as, for example, formaldehyde are vaporized and used in a above-mentioned cleaning process or a sterilization process.

### PRIOR ART LITERATURE

### Patent Document

Patent Document 1: Japanese Unexamined Patent Application Publication No. 2000-217894

### SUMMARY OF THE INVENTION

### Problem to be Solved by the Invention

The present invention was made to solve the above-mentioned problems, and a main object of this invention is to make it easier to identify the factor in case that there is a difference between an actual concentration of a compound gas made by vaporizing a compound and a desired ideal concentration.

### Means to Solve the Problems

More specifically, a gas analysis device in accordance with this invention is a gas analysis device for analyzing a compound gas and H₂O gas produced in a main reaction in which an aqueous solution comprising a compound and water are mixed to vaporize, and is characterized by comprising a first concentration calculating unit that calculates a concentration of the compound gas, a second concentration calculating unit that calculates a concentration of the H₂O gas, an analysis unit that compares a first actual concentration which is the concentration of the compound gas calculated by the first concentration calculating unit with a first ideal concentration which is the concentration of the compound gas in case that the main reaction proceeds ideally and that compares a second actual concentration which is the concentration of the H₂O gas calculated by the second concentration calculating unit with a second ideal concentration which is the concentration of the H₂O gas in case that the main reaction proceeds ideally and an output unit that outputs an analysis result based on the comparison by the analysis unit.

In accordance with the gas analysis device having the above-mentioned arrangement, since the first actual concentration and the first ideal concentration, which are the concentrations of the compound gas, are compared and the analysis results are output, similar to a conventional arrangement, it is possible to grasp whether there is a difference between the first actual concentration and the first ideal concentration or not. In addition, since the second actual concentration and the second ideal concentration, which are the concentrations of H₂O gas, are also compared and the analysis results are output, it becomes easier to identify a factor that cannot be identified only by comparing the first actual concentration and the first ideal concentration as a factor that may cause a difference in the first actual concentration and the first ideal concentration.

In case that the analysis unit judges that the first actual concentration is lower than the first ideal concentration, it is preferable that the analysis unit judges a type of a side reaction by comparing the second actual concentration with the second ideal concentration and the judged result is output by the output unit as the analysis result.

In accordance with this arrangement, it becomes easier to identify the type of the side reaction and to take appropriate measures to reduce the difference between the first actual concentration and the first ideal concentration.

More concretely, it is preferable that the type of the side reaction includes at least one of liquefaction of the compound gas, decomposition of the compound gas, and redissolution of the compound gas into a liquefied H₂O gas.

It is preferable that the analysis unit judges whether the side reaction that is different from the main reaction is occurring or not by comparing the first actual concentration with the first ideal concentration and the judged result is output by the output unit as the analysis result.

In accordance with this arrangement, in case that there is a difference between the first actual concentration and the first ideal concentration, it is possible to judge whether there is a high probability that a side reaction other than the main reaction is taking place or not, or whether there is another factor or not.

It is preferable that the analysis unit judges whether an abnormality is occurring on the side of the gas analysis device or not by comparing the first actual concentration with the first ideal concentration and the judged result is output by the output unit as the analysis result.

In accordance with this arrangement, in case that there is a difference between the first actual concentration and the first ideal concentration, it is possible to judge whether there is a high probability that an abnormality is occurring on the side of the gas analysis device side, or whether there is another factor or not.

In order to reduce the difference between the first actual concentration and the first ideal concentration or the difference between the second actual concentration and the second ideal concentration, it is preferable to further comprise an adjusting unit that adjusts a set temperature of a vaporizer that vaporizes the aqueous solution or a set flow rate of a flow control device that controls a flow rate of a fluid introduced into the vaporizer or discharged from the vaporizer.

As a more concrete embodiment represented is that the first concentration calculating unit calculates the concentration of hydrogen peroxide, formaldehyde or peracetic acid.

It is preferable that the first concentration calculating unit and the second concentration calculating unit calculate concentrations based on an output signal output from a common photodetector.

In accordance with this arrangement, since it is possible to calculate the concentration of the compound gas and the H₂O gas using the common photodetector, it is possible to downsize the gas analysis device and to reduce a manufacturing cost. In addition, a fluid control system comprising a vaporizer for vaporizing the aqueous solution, a fluid control device arranged in a flow channel which leads the aqueous solution to the vaporizer and the above-mentioned gas analysis device is one of this invention.

Furthermore, a program used for a gas analysis device in accordance with this invention is a program used for a gas analysis device that analyzes a compound gas and H₂O gas produced in a main reaction in which an aqueous solution comprising a compound and water are mixed to vaporize, and is characterized by making a computer to produce functions as a first concentration calculating unit that calculates a concentration of the compound gas, a second concentration calculating unit that calculates a concentration of the H₂O gas, an analysis unit that compares a first actual concentration, which is the concentration of the compound gas calculated by the first concentration calculating unit, with a first ideal concentration, which is the concentration of the compound gas in case that the main reaction proceeds ideally, and that compares a second actual concentration, which is the concentration of the H₂O gas calculated by the second concentration calculating unit, with a second ideal concentration, which is the concentration of the H₂O gas in case that the main reaction proceeds ideally, and an output unit that outputs an analysis results based on the comparison by the analysis unit.

In addition, a gas analysis method in accordance with this invention is a gas analysis method for analyzing a compound gas and H₂O gas produced in a main reaction in which an aqueous solution comprising a mixture of a compound and water vaporizes, and comprises an analysis step that compares a first actual concentration, which is the calculated concentration of the compound gas, with a first ideal concentration, which is the concentration of the compound gas in case that the main reaction proceeds ideally, and that compares a second actual concentration, which is the calculated concentration of the H₂O gas, with a second ideal concentration, which is the concentration of the H₂O gas in case that the main reaction proceeds ideally, and an output step that outputs an analysis results based on the comparison by the analysis step.

In accordance with the program for gas analysis and the gas analysis method, the same effect and operation can be produced as those of the above-mentioned gas analysis device.

In addition, a gas analysis device in accordance with this invention is a gas analysis device for analyzing a compound gas and H₂O gas produced in the main reaction in which an aqueous solution comprising a compound and water are mixed to vaporize, and is characterized by comprising a first concentration calculating unit that calculates a concentration of the compound gas, a second concentration calculating unit that calculates a concentration of the H₂O gas, and an output unit that outputs a first actual concentration, which is the concentration of the compound gas calculated by the first concentration calculating unit, with a first ideal concentration, which is the concentration of the compound gas in case that the main reaction proceeds ideally in a comparable manner, and that outputs a second actual concentration, which is the concentration of the H₂O gas calculated by the second concentration calculating unit, with a second ideal concentration, which is the concentration of the H₂O gas in case that the main reaction proceeds ideally, in a comparable manner.

In accordance with this arrangement, since the first actual concentration and the first ideal concentration, which are the concentrations of the compound gas, are output in a comparable manner, similar to a conventional arrangement, it is possible to grasp whether there is a difference between the first actual concentration and the first ideal concentration or not. Furthermore, since the second actual concentration and the second ideal concentration, which are the concentrations of the H₂O gas, are also output in a comparable manner, it becomes easier to specify a factor that cannot be identified only by comparing the first actual concentration and the first ideal concentration, as a factor that may cause a difference in the first actual concentration and the first ideal concentration.

### Effects of the Invention

In accordance with the above-mentioned invention, in case that there is a difference between the actual concentration and the desired ideal concentration of the compound gas made by vaporizing a compound, it becomes easier to identify the factor to cause the difference.

### BRIEF DESCRIPTION OF THE DRAWINGS

FIG. 1 A schematic diagram showing a fluid control system incorporating a gas analysis device in accordance with one embodiment of the present claimed invention.

FIG. 2 A diagram showing a chemical reaction equation to explain types of side reactions in this embodiment.

FIG. 3 A schematic diagram showing a configuration of a concentration monitor of the embodiment.

FIG. 4 A functional block diagram explaining functions of an information processing unit of this embodiment.

FIG. 5 A flowchart diagram explaining an operation of an information processing unit of this embodiment.

FIG. 6 A functional block diagram explaining functions of the information processing unit of other embodiments.

FIG. 7 A functional block diagram explaining functions of the information processing unit of the other embodiment.

FIG. 8 A schematic diagram of a fluid control system incorporating the gas analysis device of the other embodiment.

FIG. 9 A schematic diagram of a sterilization processing system incorporating the gas analysis device of the other embodiment.

### EXPLANATION OF THE CODE

100 . . . gas analysis device
200 . . . fluid control system
S . . . gas supply space
10 . . . vaporizer
L1 . . . gas supply channel
30 . . . concentration monitor
40 . . . information processing unit
41 . . . first concentration calculation unit
42 . . . second concentration calculation unit
43 . . . ideal concentration storage unit
44 . . . analysis unit
45 . . . output unit

### Modes for Embodying the Invention

A gas analysis device in accordance with this embodiment of the present claimed invention will be explained with reference to drawings.

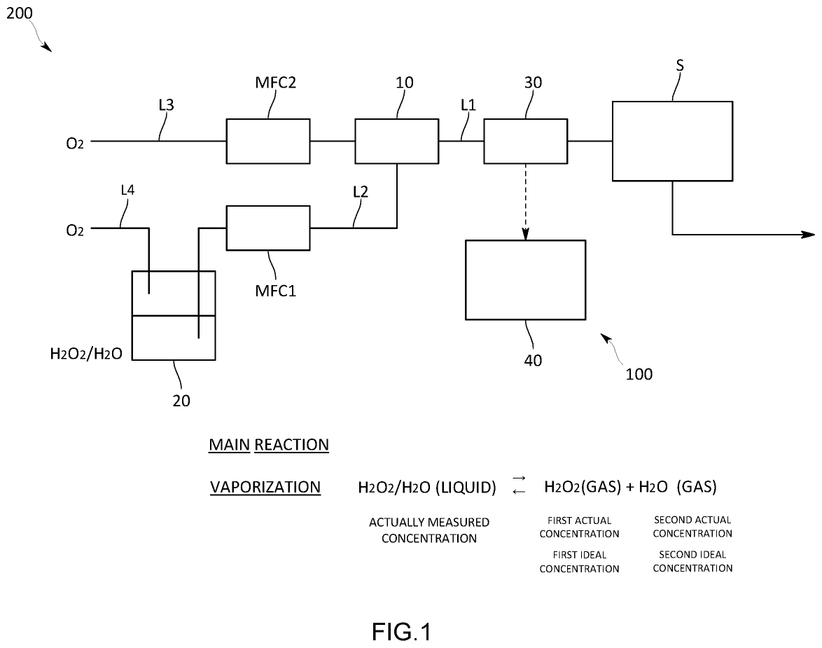

The gas analysis device 100 of this embodiment, as shown in FIG. 1, is to construct a fluid control system 200 that controls a gas supplied to a predetermined gas supply space (S) and to measure a concentration of the gas.

First, the fluid control system 200 will be explained. As shown in FIG. 1, the fluid control system 200 supplies a material gas to a process chamber which is the gas supply space (S) of, for example, a semiconductor manufacturing device. Concretely, the fluid control system 200 comprises a vaporizer 10 that vaporizes an aqueous solution made of a mixture of a compound and water as a liquid material, and a gas supply channel L1 that supplies the material gas, which is made by vaporizing the liquid material by the vaporizer 10, to the process chamber as the gas supply space (S). The liquid material in this embodiment is a mixture of the hydrogen peroxide (H₂O₂) and water (H₂O) in which a concentration of the hydrogen peroxide is adjusted to a desired concentration, and the material gas is the hydrogen peroxide gas.

The vaporizer 10 heats and/or depressurizes the liquid material to vaporize the liquid material and comprises a heater (not shown in drawings) to heat the liquid material and a nozzle (not shown in drawings) to jet and vaporize the liquid material. The vaporizer 10 is connected to a material introduction channel L2 through which the liquid material stored in a reservoir 20 is led and a carrier gas introduction channel L3 through which the carrier gas is led, and the reservoir 20 is connected to a pumping gas introduction channel LA through which a pumping gas is led. In addition, the material introduction channel L2 is provided with a first mass flow controller MFC1, which is a fluid control device that controls the flow rate of the liquid material, and the carrier gas introduction channel L3 is provided with a second mass flow controller MFC2, which is a fluid control device that controls the flow rate of the carrier gas. Although oxygen is used in this embodiment as the carrier gas and the ‘pumping gas, nitrogen, argon, or hydrogen may be used depending on the type of the liquid material.

As shown in FIG. 1, the gas supply channel L1 connects the vaporizer 10 and the gas supply space (S). The compound gas and its byproduct gas, which are produced by the main reaction of vaporizing the aqueous solution consisting of a mixture of compound and water, flow through the gas supply channel L1. In this embodiment, the compound gas is a hydrogen peroxide gas and the byproduct gas is a H₂O gas, and the carrier gas and oxygen, which is the above-mentioned pumping gas, also flow in the gas supply channel L1 together with the compound gas and the byproduct gas.

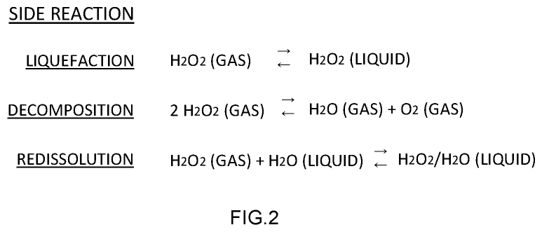

In this embodiment, the byproduct gas is produced by the main reaction as described above, however, the byproduct gas is also produced by the side reactions so that the concentration of the byproduct gas can also fluctuate due to the side reaction, and the byproduct gas can also be a cause of fluctuation in the concentration of the compound gas. Then, the present claimed invention finds a technical significance of monitoring the concentration of the biproduct gas and the detail of this invention will be described below. The side reaction in this embodiment includes, as shown in FIG. 2, the liquefaction of the hydrogen peroxide gas, the decomposition of the hydrogen peroxide gas, and the re-dissolution of the hydrogen peroxide gas in water which is the liquefied H₂O gas.

As shown in FIG. 1, the gas analysis device 100 of this embodiment comprises a concentration monitor 30 arranged in the gas supply channel L1 and an information processing unit 40 that acquires output signals from the concentration monitor 30. The concentration monitor 30 is not necessarily provided in the gas supply channel L1, however, may be provided, for example, in a branch flow channel branched from the gas supply channel L1.

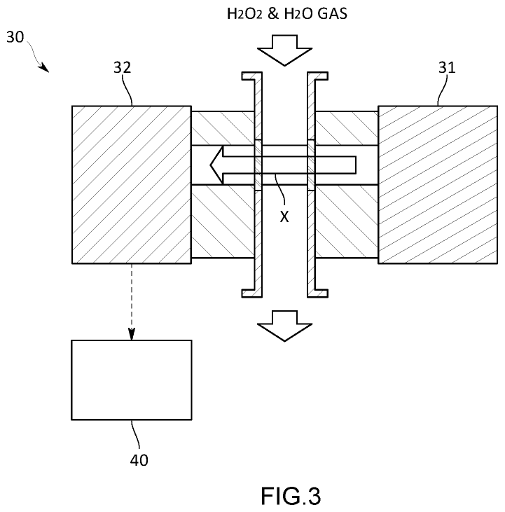

The concentration monitor 30 analyzes the component to be measured contained in the gas by the infrared absorption method, and as shown in FIG. 3, concretely comprises a light source unit 31 housing a light source that irradiates the infrared light (X) on the gas, and a detection unit 32 housing a photo detector that detects the infrared light (X) that has penetrated the gas. A light intensity signal of the infrared light (X) detected by the photodetector is output to the information processing unit 40 as an output signal.

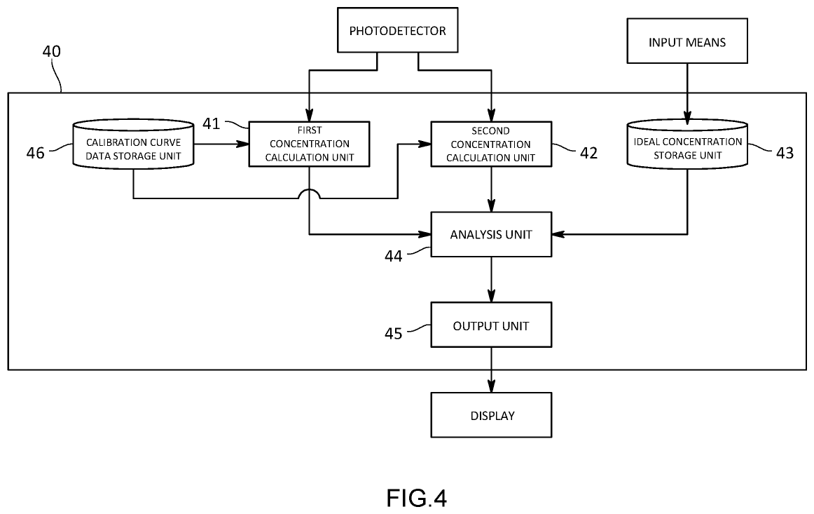

The information processing unit 40 is a general-purpose or dedicated computer comprising a CPU, a memory, an AD converter, a DA converter or the like, and may be integrated with the concentration monitor 30 or may be separated from the concentration monitor 30. The information processing unit 40 performs functions as a first concentration calculation unit 41, a second concentration calculation unit 42, an ideal concentration storage unit 43, an analysis unit 44 and an output unit 45, as shown in FIG. 4, in cooperation with the CPU and its peripheral devices in accordance with gas analysis programs stored in a predetermined area of the memory. The concentration of a gas to be described below may mean the concentration of the component of the gas or the partial pressure of the gas.

An operation of the information processing unit 40 in this embodiment will be also explained below, along with an explanation of the functions of each unit.

The first concentration calculation unit 41 calculates the concentration (hereinafter also referred to as the first actual concentration) of the hydrogen peroxide gas which is the compound gas. Concretely, the first concentration calculation unit 41 receives the light intensity signal which is the output signal from the photo detector and conducts an arithmetic process on the value indicated by the light intensity signal to calculate the concentration of the hydrogen peroxide gas contained in the gas flowing in the gas supply channel L1 as the first actual concentration. The first calibration curve data indicating a relationship between the value indicated by the light intensity signal and the first actual concentration is used in the arithmetic process, and the first calibration curve data is stored in a calibration curve data storage unit 46 set in a predetermined area of the memory (refer to FIG. 4).

The second concentration calculation unit 42 calculates the concentration (hereinafter also referred to as the second actual concentration) of the H₂O gas which is the byproduct gas. Concretely, the second concentration calculation unit 42 receives the light intensity signal which is the output signal from the photo detector and conducts an arithmetic process on the value indicated by the light intensity signal to calculate the concentration of the H₂O gas contained in the gas flowing in the gas supply channel L1 as the second actual concentration. The second calibration curve data which indicates a relationship between the value indicated by the light intensity signal and the second actual concentration is used in the arithmetic process, and the second calibration curve data is stored in the calibration curve data storage unit 46 set in the predetermined area of the memory (refer to FIG. 4).

In this embodiment, the first concentration calculation unit 41 and the second concentration calculation unit 42 are configured to calculate the first actual concentration and the second actual concentration respectively based on the output signals output from a common photo detector, thereby downsizing the gas analysis device 100 and reducing a manufacturing cost. However, the first concentration calculation unit 41 and the second concentration calculation unit 42 may be configured to calculate the first actual concentration and the second actual concentration respectively based on output signals output from different photo detectors respectively.

The ideal concentration storage unit 43 is set in a predetermined area of the memory and stores the first ideal concentration, which is the concentration of the hydrogen peroxide gas in case that the above-mentioned main reaction proceeds ideally, and the second ideal concentration, which is the concentration of the H₂O gas in case that the above-mentioned main reaction also proceeds ideally.

The first ideal concentration can be obtained by calculating in advance before starting a control process, for example, by the fluid control system 200. Concretely, the first ideal concentration can be obtained based on a theoretical concentration of the hydrogen peroxide (concretely a volume fraction of the hydrogen peroxide) which is theoretically obtained by using a titration concentration obtained by actually measuring the concentration of the hydrogen peroxide contained in the aqueous solution stored in the reservoir 20 by titration, and a total flow rate of the gas flowing through the concentration monitor 30 (a total flow rate of the hydrogen peroxide gas, the H₂O gas and oxygen gas). The theoretical concentration is the concentration of the hydrogen peroxide gas in case that 100% of the aqueous solution stored in the reservoir 20 is vaporized, in other words, the concentration of the hydrogen peroxide gas in case that only the above-mentioned main reaction is occurring. The theoretical concentration can be used as the first ideal concentration, however, the first ideal concentration in this embodiment is set in consideration of the fact that the compound gas decreases considerably due to condensation in a process of reaching from the reservoir 20 to the concentration monitor 30. In other words, since there is a difference between the theoretical concentration and the concentration (referred to an effective concentration) measured by the concentration monitor 30, a ratio (hereinafter referred to as vaporization efficiency) of the effective concentration to the theoretical concentration is obtained in advance, and the concentration obtained by multiplying the vaporization efficiency by the theoretical concentration is used as the first ideal concentration. The effective concentration may be used as the first ideal concentration without obtaining the vaporization efficiency.

The second ideal concentration, similar to the first ideal concentration, can be obtained in advance before starting the control process, for example, by the fluid control system 200. Concretely, the second ideal concentration can be calculated based on the theoretical concentration of H₂O (concretely, the volume fraction of H₂O) which is theoretically obtained using the above-mentioned titration concentration and the total flow rate of the gas flowing in the concentration monitor 30, and the concentration obtained by multiplying the theoretical concentration by the above-mentioned vaporization efficiency is used as the second ideal concentration in this embodiment. The concentration of the H₂O gas measured in advance by the concentration monitor 30 before starting the control process by the fluid control system 200 may also be used as the second ideal concentration.

The first ideal concentration and the second ideal concentration calculated in this manner are input from outside through, for example, input means, and stored in the ideal concentration storage unit 43. However, the information processing unit 40 may be provided with a function as an ideal concentration calculation unit to calculate the first ideal concentration and the second ideal concentration, and the first ideal concentration and the second ideal concentration calculated by the ideal concentration calculation unit may be stored in the ideal concentration storage unit 43.

The analysis unit 44 compares the first actual concentration and the first ideal concentration and compares also the second actual concentration and the second ideal concentration. Concretely, the analysis unit 44 judges a size relation of the first actual concentration and the first ideal concentration and judges also the size relation of the second actual concentration and the second ideal concentration.

The analysis unit 44 of this embodiment is configured to determine whether the side reaction other than the main reaction is occurring or not by comparing the first actual concentration and the first ideal concentration, as well as to determine whether an abnormality is occurring on the side of the gas analysis device 100 or not.

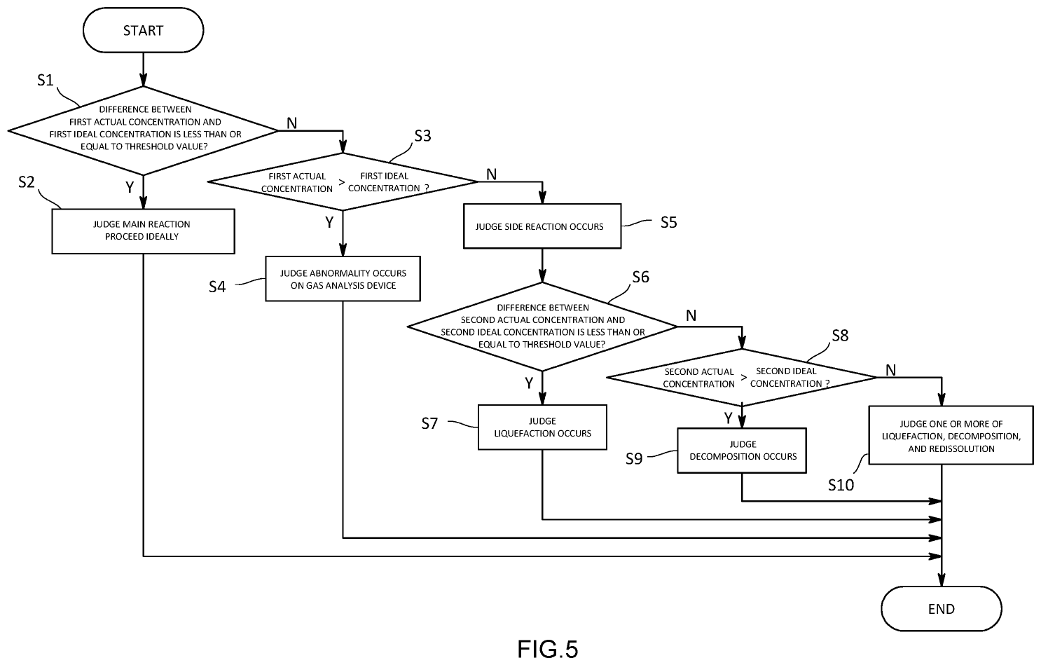

More concretely, as shown in FIG. 5, the analysis unit 44 first compares the first actual concentration and the first ideal concentration (S1). In case that the difference between the first actual concentration and the first ideal concentration is less than or equal to a predetermined threshold value, the analysis unit 44 judges that the above-mentioned main reaction proceeds ideally (S2).

On the other hand, if the difference between the first actual concentration and the first ideal concentration exceeds the predetermined threshold value in (S1), the analysis unit 44 judgess the size relation between the first actual concentration and the first ideal concentration (S3) and judges whether the side reaction other than the main reaction is occurring or not, or whether an abnormality is occurring on the side of the gas analysis device 100 or not (S4, S5).

Concretely, in case that the first actual concentration is higher than the first ideal concentration, the analysis unit 44 judges that an abnormality is occurring on the side of the gas analysis device 100 (S4). The abnormality can be, for example, a calibration failure or a setting error in various setting values such as the first calibration curve data, the second calibration curve data, the vaporization efficiency or the like.

In contrast, in case that the first actual concentration is lower than the first ideal concentration, the analysis unit 44 judges that the side reaction other than the main reaction is occurring (S5).

In case that it is judged that a side reaction is occurring in S5, the analysis unit 44 identifies a type of the side reaction based on the results of the comparison between the second actual concentration and the second ideal concentration. As mentioned above, the types of the side reactions include liquefaction of the hydrogen peroxide gas, decomposition of the hydrogen peroxide gas, and redissolution of the hydrogen peroxide gas into water in which the H₂O gas is liquefied (refer to FIG. 2). The type of the side reaction identified by the analysis unit 44 should include at least one of the liquefaction, decomposition and redissolution.

The analysis unit 44 in this embodiment compares the second actual concentration with the second ideal concentration (S6), and in case that the difference between the second actual concentration and the second ideal concentration is less than a predetermined threshold value, the analysis unit 44 judges that liquefaction of the hydrogen peroxide gas is occurring as the side reaction (S7).

On the other hand, in case that the difference between the second actual concentration and the second ideal concentration exceeds the predetermined threshold value in S6, the analysis unit 44 judges the size relation between the second actual concentration and the second ideal concentration (S8). In case that the second actual concentration is higher than the second ideal concentration, the analysis unit 44 judges that decomposition of the hydrogen peroxide gas is occurring as the side reaction (S9), and in case that the second actual concentration is lower than the second ideal concentration, the analysis unit 44 judges that one or more of liquefaction of the hydrogen peroxide gas are occurring as the side reactions (S10).

As mentioned above, the analysis results by the analysis unit 44 include at least the result of the comparison between the first actual concentration and the first ideal concentration and the result of the comparison between the second actual concentration and the second ideal concentration. Furthermore, the analysis results in this embodiment also include various judgment results based on those comparison results; more specifically, whether there is an abnormality on the side of the gas analysis device 100 or not, or whether the side reaction other than the main reaction is occurring or not and the type of the occurring side reaction (liquefaction, decomposition, or re-dissolution).

The analysis results based on the comparison by the analysis unit 44 are visibly output by the output unit 45. Concretely, the output unit 45 visibly outputs some or all of the information included in the analysis results and is configured to display and output on the display the fact that there is the abnormality on the side of the gas analysis device 100, that the side reaction is occurring, and the type of the side reaction. The output unit 45 may also be used to print out the analysis results on paper or other media.

In accordance with the gas analysis device 100 of this embodiment having the above-mentioned arrangement, since the first actual concentration and the first ideal concentration, which are the concentrations of the hydrogen peroxide gas, are compared and the analysis results are output, it is possible to grasp whether there is the difference between the first actual concentration and the first ideal concentration, more specifically, whether the main reaction proceeds ideally.

In addition, since the second actual concentration and the second ideal concentration, which are the concentrations of the H₂O gas, are also compared and the analysis results are output, it becomes easier to specify a highly provable factor among various factors, which cannot be determined only by comparing the first actual concentration and the first ideal concentration, such as occurrence of the abnormality and occurrence of the side reaction such as liquefaction, decomposition, or redissolution of the hydrogen peroxide gas, as a factor causing the difference between the first actual concentration and the first ideal concentration, and thus it is easier to take appropriate measures to reduce the difference between the first actual concentration and the first ideal concentration.

The present invention is not limited to the above-mentioned embodiment.

For example, the output unit 45 of the above-mentioned embodiment outputs that there is the abnormality on the side of the gas analysis device 100, the side reaction is occurring and the type of the occurring side reaction, however, the output unit 45 may output only some of them. In addition, the comparison results (size relation) between the first actual concentration and the first ideal concentration and the comparison results (size relation) between the second actual concentration and the second ideal concentration may also be displayed. In this case, the analysis unit 44 does not need to determine that there is an abnormality on the side of the gas analysis device 100, a side reaction is occurring, and even the type of the side reaction.

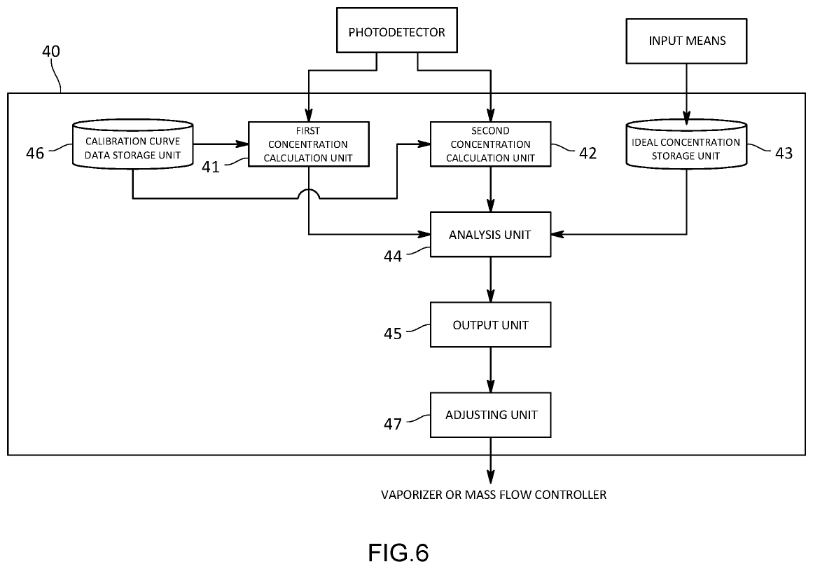

Furthermore, the output unit 45 may output the analysis results to the adjusting unit 47, as shown in FIG. 6, in addition to displaying or printing out the analysis results. The adjusting unit 47 may be configured to adjust, for example, the set temperature of the vaporizer 10 and the set flow rates of the mass flow controllers MFC1 and MFC2 so as to reduce the difference between the first actual concentration and the first ideal concentration.

In addition, the output unit 45 may be configured to output the first actual concentration and the first ideal concentration in a comparable manner and to output the second actual concentration and the second ideal concentration in a comparable manner for example, on a display without outputting the analysis results by the analysis unit 44. In this case, the information processing unit 40 does not need to have a function as the analysis unit 44.

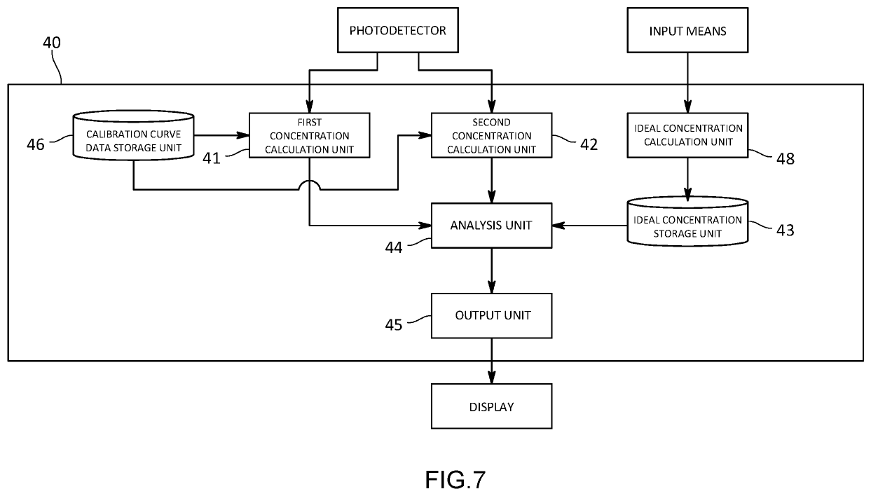

Furthermore, as shown in FIG. 7, as mentioned in the above-mentioned embodiment, the information processing unit 40 may also be provided with a function as an ideal concentration calculation unit 48 that calculates the first ideal concentration and the second ideal concentration. Concretely, as the ideal concentration calculation unit 48 represented is a form of calculating the first ideal concentration and the second ideal concentration using the vaporization efficiency input through an input means.

The information processing unit 40 may be further provided with a function as a reporting unit that reports when the difference between the first actual concentration and the first ideal concentration exceeds a predetermined threshold value as a result of the comparison unit comparing the first actual concentration and the first ideal concentration.

Moreover, some of the functions of the information processing unit 40 as the first concentration calculation unit 41, the second concentration calculation unit 42, the analysis unit 44 and the output unit 45 may be provided by another computer, or the ideal concentration storage unit 43 may be set in a predetermined area of an external memory different from the memory of the information processing unit 40.

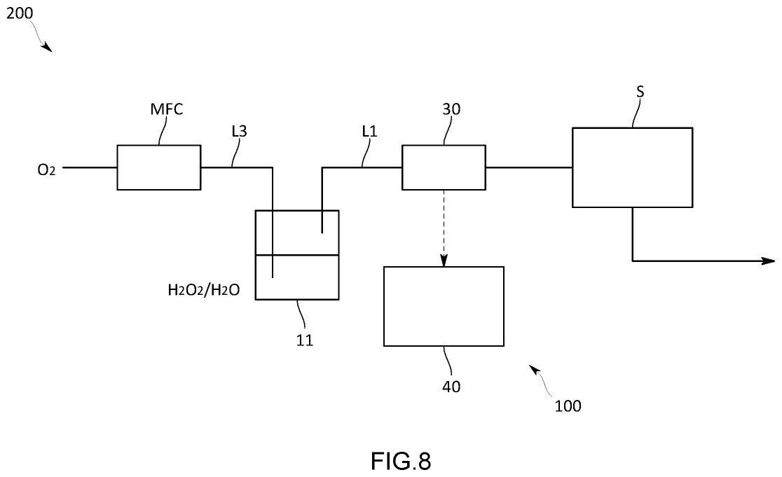

The fluid control system 200 of the above-mentioned embodiment vaporizes the liquid material by jetting the liquid material out with a nozzle, however, the fluid control system 200 may vaporize a liquid material by applying heat to the liquid material to bubble, as shown in FIG. 8.

Concretely, the fluid control system 200 comprises a vaporizer provided with a vaporization tank 11 that houses an aqueous solution consisting of a mixture of a compound and water and that vaporizes the aqueous solution, a carrier gas introduction channel L3 that introduces a carrier gas into the vaporization tank 11, a mass flow controller (MFC) which is a fluid control device arranged in the carrier gas introduction channel L3 and a gas supply channel L1 that supplies the gas vaporized by the vaporization tank 11 to a gas supply space (S) such as a chamber, and further comprises a concentration monitor 30 arranged in the gas supply channel L1 and an information processing unit 40 that acquires output signals from the concentration monitor 30.

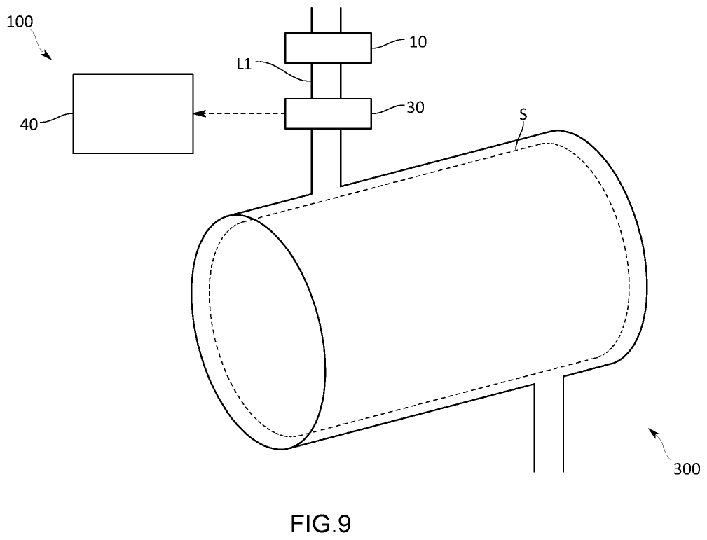

In addition, the gas analysis device 100 of the present claimed invention may also be applied to a sterilization processing apparatus 300 for sterilizing, for example, an object to be sterilized such as medical equipment as shown in FIG. 9.

Concretely, the sterilization processing apparatus 300 comprises a gas supply space (S), which is a chamber that houses the object to be sterilized, a vaporizer 10 that vaporizes an aqueous solution consisting of a mixture of a compound and water, and a gas supply channel L1 that leads the gas vaporized by the vaporizer 10 into the chamber (S), and further comprises a concentration monitor 30 arranged in the gas supply channel L1 and an information processing unit 40 that acquires output signals from the concentration monitor 30.

In addition, the compound to be mixed with water may be formaldehyde, although hydrogen peroxide is used as an example in the above-mentioned embodiment. In other words, the first concentration calculation unit 41 may be used to calculate the concentration of formaldehyde contained in the gas flowing in the gas supply channel L1.

Furthermore, the compound to be mixed with water may be peracetic acid. In this case, a concrete embodiment may be that an aqueous solution of peracetic acid mixed with water is contained in a container, and the concentration of the peracetic acid gas and the H₂O gas contained in the vapor in the container may be monitored by the concentration monitor 30.

In addition, various other modifications and combinations of embodiments may be possible without departing from the spirit of the invention.

### POSSIBLE APPLICATIONS IN INDUSTRY

In accordance with the present invention, in case that there is a difference between an actual concentration of a compound gas made by vaporizing a compound and a desired ideal concentration thereof, it becomes easier to identify a cause of the difference.

1. A gas analysis device for analyzing a compound gas and H₂O gas produced in a main reaction in which an aqueous solution comprising a compound and water are mixed to vaporize, comprising
a first concentration calculating unit that calculates a concentration of the compound gas,
a second concentration calculating unit that calculates a concentration of the H₂O gas,
an analysis unit that compares a first actual concentration which is the concentration of the compound gas calculated by the first concentration calculating unit with a first ideal concentration which is the concentration of the compound gas in case that the main reaction proceeds ideally, and that compares a second actual concentration which is the concentration of the H₂O gas calculated by the second concentration calculating unit with a second ideal concentration which is the concentration of the H₂O gas in case that the main reaction proceeds ideally and
an output unit that outputs an analysis result based on the comparison by the analysis unit.

2. The gas analysis device described in claim 1, wherein
in case that the analysis unit judges that the first actual concentration is lower than the first ideal concentration, the analysis unit judges a type of a side reaction by comparing the second actual concentration with the second ideal concentration and
the judged result is output by the output unit as the analysis result.

3. The gas analysis device described in claim 2, wherein
the type of the side reaction includes at least one of liquefaction of the compound gas, decomposition of the compound gas, and redissolution of the compound gas into liquefied H₂O gas.

4. The gas analysis device described in claim 1, wherein
the analysis unit judges whether the side reaction that is different from the main reaction is occurring or not by comparing the first actual concentration with the first ideal concentration and
the judged result is output by the output unit as the analysis result.

5. The gas analysis device described in claim 1, wherein
the analysis unit judges whether an abnormality is occurring on the side of the gas analysis device or not by comparing the first actual concentration with the first ideal concentration and
the judged result is output by the output unit as the analysis result.

6. The gas analysis device described in claim 1, further comprising
an adjusting unit that adjusts a set temperature of a vaporizer that vaporizes the aqueous solution or a set flow rate of a flow control device that controls a flow rate of a fluid introduced into the vaporizer or discharged from the vaporizer.

7. The gas analysis device described in claim 1, wherein
the first concentration calculating unit calculates the concentration of hydrogen peroxide, formaldehyde or peracetic acid.

8. The gas analysis device described in claim 1, wherein
the first concentration calculating unit and the second concentration calculating unit calculate concentrations based on an output signal output from a common photodetector.

9. A fluid control system comprising
a vaporizer for vaporizing the aqueous solution,
a fluid control device arranged in a flow channel which leads the aqueous solution to the vaporizer and
the gas analysis device described in claim 1.

10. A computer-readable medium including a program used for a gas analysis device that analyzes a compound gas and H₂O gas produced in a main reaction in which an aqueous solution comprising a compound and water are mixed to vaporize, wherein making a computer to produce functions as
a first concentration calculating unit that calculates a concentration of the compound gas,
a second concentration calculating unit that calculates a concentration of the H₂O gas,
an analysis unit that compares a first actual concentration which is the concentration of the compound gas calculated by the first concentration calculating unit with a first ideal concentration which is the concentration of the compound gas in case that the main reaction proceeds ideally, and that compares a second actual concentration which is the concentration of the H₂O gas calculated by the second concentration calculating unit with a second ideal concentration which is the concentration of the H₂O gas in case that the main reaction proceeds ideally and
an output unit that outputs an analysis results based on the comparison by the analysis unit.

11. A gas analysis method for analyzing a compound gas and H₂O gas produced in a main reaction in which an aqueous solution comprising a mixture of a compound and water vaporizes, comprising
analyzing that compares a first actual concentration which is the calculate concentration of the compound gas with a first ideal concentration which is the concentration of the compound gas in case that the main reaction proceeds ideally, and that compares a second actual concentration which is the calculated concentration of the H₂O gas with a second ideal concentration which is the concentration of the H₂O gas in case that the main reaction proceeds ideally and
outputting an analysis results based on the comparison by the analyzing.

12. A gas analysis device for analyzing a compound gas and H₂O gas produced in the main reaction in which an aqueous solution comprising a compound and water are mixed to vaporize, comprising
a first concentration calculating unit that calculates a concentration of the compound gas,
a second concentration calculating unit that calculates a concentration of the H₂O gas and
an output unit that outputs a first actual concentration which is the concentration of the compound gas calculated by the first concentration calculating unit with a first ideal concentration which is the concentration of the compound gas in case that the main reaction proceeds ideally in a comparable manner, and that outputs a second actual concentration which is the concentration of the H₂O gas calculated by the second concentration calculating unit with a second ideal concentration which is the concentration of the H₂O gas in case that the main reaction proceeds ideally in a comparable manner.
